(19) United States
(12) Patent Application Publication
(10) Pub. No.: US 2006/0057083 A1
(43) Pub. Date: Mar. 16, 2006

(54) Cosmetic composition comprising at least one diamantoid to improve the mechanical properties of some materials

(76) Inventor: Estelle Mathonneau, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/220,717

(22) Filed: Sep. 8, 2005

Related U.S. Application Data
(60) Provisional application No. 60/621,341, filed on Oct. 22, 2004.

(30) Foreign Application Priority Data
Sep. 9, 2004 (FR) 04 52008

(51) Int. Cl. A61K 8/31

(52) U.S. Cl. 424/61; 424/401; 424/64

(57) ABSTRACT

The present disclosure relates to a cosmetic composition comprising, in a cosmetically acceptable medium, at least one diamantoid comprising at least two adamantane units. The present disclosure further relates to a method for treating keratin materials, as well as a method for improving the mechanical properties of some materials that are used in cosmetics.

This application claims benefit of U.S. Provisional Application No. 60/620,341, filed Oct. 21, 2004, the contents of which are incorporated herein by reference. This application also claims benefit of priority under 35 U.S.C. § 119 to French Patent Application No. 04 52008, filed Sep. 9, 2004, the contents of which are also incorporated herein by reference.

The present disclosure relates to novel cosmetic compositions, comprising at least one diamantoid, as well as methods for treating keratin materials comprising applying a cosmetic composition comprising at least one diamantoid.

Many hair styling products for fixing the hair comprise either hard or soft polymers. There is a constant need in the art for the improvement of some of the mechanical properties of these polymers. Many material types have been employed to this end, the most current of which are fillers.

In the field of make-up and skin care, there are also many filler-based combinations aimed at improving the mechanical properties of the polymers that are used in the compositions. These fillers are most often inorganic in nature and silica is the most often used filler.

Moreover, most hair styling products intended to add volume to the hair are based on film-forming polymers. Possible drawbacks associated with the use of film-forming polymers are, for example, that the cosmetic effect may immediately disappear after the first shampoo, and that the hair feel may be affected (e.g., the hair may feel heavy). Also known are methods for permanently treating the keratin fibers wherein such method provide a feel that is not so heavy. These treatments employ a reducing agent and an oxidizing agent and require a mechanical tensioning of the hair by means of rolling material so as to give the hair a long lasting shape.

Although they can effectively increase the hair volume, the methods discussed above can suffer from the drawbacks of being harmful to the hair shape and/or to the frizz volume, and/or of damaging the nature of the keratin fiber. It has been proposed to use carbon nanotubes so as to increase hair volume without thereby making the hair feel heavier, for example, in French Patent Application Publication No. FR 2 840 529. These nanotubes are useful in that they may not cause an increase in the viscosity of the medium. Nevertheless, the nanotubes may be sometimes difficult to move on the substrates and the cosmetic vehicle has to be carefully selected, thus sometimes limiting the potential application types. Moreover the nanotubes can be difficult to functionalize.

Thus, there is a need to develop compositions to improve the mechanical properties of some materials that are used in cosmetics without thereby affecting the appearance of the composition, and, for instance, without making the hair feel heavy.

Accordingly, the present disclosure relates to novel cosmetic compositions which can limit and/or eliminate some or all of such drawbacks.

More specifically, the inventor has discovered, surprisingly, that a composition comprising at least one diamantoid can improve the mechanical properties of some materials, such as polymers, that are used in cosmetics, such as in the fields of make-up, skin care, hair care, hair styling and coloring or body care products.

Thus the present disclosure relates to a cosmetic composition comprising at least one diamantoid.

The present disclosure further relates to a method for treating keratin materials, comprising applying a cosmetic composition such as defined herein.

Other aspects of the present disclosure will become clear from the following description, and in view of the following non-limiting example.

The present disclosure relates to a cosmetic composition comprising, in a cosmetically acceptable medium, at least one diamantoid comprising at least two adamantane units.

As used herein, the term “diamantoid” is understood to mean a nanometric sized hydrocarbide in the solid state. The most elemental diamantoid comprises 10 carbon atoms and is a (C₁₀H₁₆) adamantane. Multiple adamantane “units” form diamantoids also called polyadamantanes, for example the pentamantane (5 units). Other diamantoid forms besides pentamantane may exist. There are, for example, helicoidal or cubic or cylindric shapes. Diamantoids have a strong rigidity since some carbon atoms have a tetragonal conformation similar to that of the carbon in diamond, but also have a good thermal stability and satisfactory transparency properties due to their size.

Classically, diamantoids may be obtained by separation from hydrocarbides such as natural gas. Diamantoid extraction methods from natural gas have been described in U.S. Pat. Nos. 5,019,665; 5,461,184; and 5,498,812. These methods comprise solubilizing the diamantoids contained in the natural gas with suitable solvents, then in separating the diamantoids from the solvent.

Adamantane units forming the diamantoids may comprise at least one heteroatom chosen from the elements of the columns III, IV, V and VI of the periodic table, except carbon.

Diamantoids comprising heteroatoms, as well as synthesis methods thereof have been described in U.S. Pat. No. 5,367,097, U.S. Patent Application Publication No. 2004/0059145, and in International Patent Application Publication Nos. WO 9428885 and WO 2004/01512.

In one embodiment of the present disclosure, diamantoids may be functionalized to obtain good solubilization or optimal properties in the envisaged cosmetic medium. As used herein, the term “functionalized” is understood to mean the presence of functional moieties that may physically or chemically interact with one another or with the exterior medium.

Any reaction mechanism may be used to functionalize adamantane units forming the diamantoids according to the present disclosure. Diamantoid functionalization may, for example, be conducted by a reaction mechanism such as a nucleophilic substitution, an electrophilic substitution, a free radical substitution, an addition, an elimination, a rearrangement, an oxidation, a reduction, an acido-basic reaction, an electrochemical reaction or a photochemical reaction.

Each adamantane unit may comprise from 1 to 10 functional moieties which can be the same or different.

Among the functional moieties that can be used to obtain functionalized diamantoids, carboxylic acid, amine amide, ester, nitrite, hydroxy, thiol, alkyl, alkenyl, aryl moieties may be mentioned in a non-limiting manner as examples. These moieties may also be chosen from oligomers, polymers and dendrimers.

If necessary, these reactions may bear substitutions on multivalent atoms.

Not only can the diamantoid dispersion into the cosmetic media be improved, but also adamantane unit functionalization can be carried out so as to increase the diamantoid affinity for the keratin material.

The increased affinity between the diamantoids and the keratin material resulting from functionalization can be due to the increased Van der Waals type interactions and/or to the creation of hydrogen and/or ionic bonds. Thus, the at least one optional functional moiety may create with some materials, such as the keratin fibers, at least one bond chosen from Van der Waals type interactions, hydrogen bonds, ionic bonds and covalent bonds.

In this context, non-limiting mention may be made of diamantoid arylation that is described in U.S. Pat. No. 5,347,063, diamantoid coupling with aromatic rings described in U.S. Pat. No. 5,369,213, and an amine functionalization, such as described in U.S. Pat. No. 5,380,947.

Functionalized diamantoids suitable for use according to the present disclosure have also been described in International Patent Application Publication Nos. WO 02/057201 and WO 03/050066.

Substituted diamantoids may also be polymerized. Substituted diamantoid polymerization reactions are described, for instance, in U.S. Pat. No. 5,053,434, and International Patent Application Publication Nos. WO 92/13909 and WO 02/057201.

To improve the diamantoid affinity for the keratin material, adamantane units may also be covalently grafted onto the keratin fiber through at least one reactive moiety.

To that end, functionalization is performed with at least one moiety having some reactivity to the amino acids forming the keratin material.

For example, the at least one functional moiety that can create at least one covalent chemical bond with the keratin fibers can be chosen from moieties that can react with thiols, disulfides, carboxylic acids, alcohols and amines.

By way of non-limiting example, the at least one functional moiety that can react with thiols, disulfides, carboxylic acids, alcohols and amines can be chosen from:
epoxides;
aziridine ring moieties;
vinyl and activated vinyl moieties, such as acrylonitrile, acrylic and methacrylic esters, crotonic acid and ester, cinnamic acids and esters, styrene and derivatives thereof, butadiene, vinyl ethers, vinyl ketones, maleic esters, maleimides, vinyl sulfones, vinyl cyanoacrylate;
carboxylic acids and derivatives thereof, such as anhydride, acid chloride, ester functional moieties;
acetals, hemiacetals;
aminals, hemiaminals;
ketones, alpha-hydroxy ketones, alpha-haloketones;
lactones, thiolactones;
isocyanate;
thiocyanate;
imines;
imides (succinimides, glutimides), such as N-hydroxysuccimide ester;
imidates;
oxazine and oxazoline;
oxazinium and oxazolinium;
alkyl or aryl or aralkyl halides, the halogen being selected from iodine, bromine or chlorine;
unsaturated ring halides, the ring being a carbon or a heterocyclic ring, such as chlorotriazine, chloropyrimidine, chloroquinoxaline, chlorobenzotriazole;
sulfonyl halides of formula RSO₂X, wherein R is an alkyl moiety and X is chosen from fluorine and chlorine; and
silicon derivatives such as alkoxy silanes and silanols.

In one embodiment of the present disclosure, for optimal diamantoid dispersion in the cosmetic medium concerned, the composition can comprise at least one surfactant.

The at least one surfactant can be chosen from amphiphilic molecules, amphiphilic oligomers, amphiphilic polymers, and mixtures thereof. This surfactant may be a non ionic, anionic, cationic or amphoteric surfactant.

The at least one diamantoid can range in size from 1 nm to 1 mm.

The at least one diamantoid can have a molecular weight, for example, ranging from 136 g/mol to 400 g/mol.

Among the various surfactant hydrophobic moieties that may be used, non-limiting mention may be made of, for instance, moieties that can absorb themselves by so called “π stating” (aromatic ring electronic clouds covering) on the surface of the adamantane units. Among the molecules that can produce such an interaction, non-limiting examples include aromatic molecules such as styrene and derivatives thereof, or pyrene and derivatives thereof.

In one embodiment of the present disclosure, the hydrophilic moieties are chosen so as to improve nanotube surface affinity for the keratin material, either by increasing the Van der Waals type interactions and/or by creating hydrogen and/or ionic bonds.

The at least one diamantoid can be present in the cosmetic composition in an amount ranging from 0.00001% to 30%, for instance from 0.0001% to 10%, such as from 0.001% to 5% by weight, relative to the total weight of the composition.

The cosmetically acceptable medium comprising at least one diamantoid can comprise, by way of non-limiting example:
water,
aliphatic or aromatic alcohols, such as ethanol, benzyl alcohol, fatty alcohols, modified or non modified polyhydric alcohols, such as glycerol, glycol, propylene glycol, dipropylene glycol, butylene glycol, butyl diglycol,
volatile or non volatile silicones,
mineral, organic or vegetal oils,
oxyethylenated or non oxyethylenated waxes, paraffins, and alkanes, for instance C₅-C₁₀ alkanes, fatty acids, fatty amines, fatty esters and for example, fatty alcohol benzoates or salicylates,
acetone, methylethylketone, methyl acetate, butyl acetate, ethyl acetate, dimethoxyethane, diethoxyethane,
and mixtures thereof.

The cosmetically acceptable medium may come as it is, or be in emulsified form or as a dispersion. The at least one diamantoid, or some agents of the composition, may be encapsulated.

The cosmetic compositions according to the present disclosure may further comprise at least one propellant, such as for use in sprays. In one embodiment of the present disclosure, the propellant comprises a compressed or liquefied gas chosen from air, carbon dioxide, compressed nitrogen, a soluble gas such as dimethyl ether, halogenated hydrocarbides, such as fluorinated or not, and mixtures thereof.

The composition may further comprise at least one cosmetic adjuvant chosen from silicones in soluble, dispersed, for instance micro- or nano-dispersed form; thickening agents; conditioning agents; softening agents; anti-foaming agents; moisturizing agents; emollient agents; plasticizers; water-soluble and/or oil-soluble, silicone and//or non silicone sunscreens; permanent and/or temporary dyes; inorganic and/or organic pigments, colored or not; inorganic fillers; clays; pearlescent agents; opacifying agents; colloids; fragrances; peptizing agents; preserving agents; ceramides and pseudo-ceramides; vitamins and provitamins including panthenol; proteins; sequestering agents; solubilizing agents; alkalinizing agents; anticorrosion agents; fats such as vegetable, animal, mineral and synthetic oils; reducing agents; antioxidants; oxidizing agents; and mixtures thereof.

The cosmetic composition as disclosed herein may be rinsed off or not and may be provided in various galenic forms.

For example, the cosmetic composition may be a composition to be applied onto the hair and may come as a lotion, a spray (aerosol or not), a foam, a gel, a paste, a cream, a stick, a shampoo, an after-shampoo (conditioner) or a hair repair product, a hair bleaching composition, a perm composition, a coloring composition, or also a defrizzing, dyeing composition.

Alternatively, the cosmetic composition may be a composition to be applied onto the skin and may come as a gel, a spray (aerosol or not), a foam, a cream, a lipstick, an eye-liner, a mascara, a blush, a nail varnish polish, a shaving foam or a powder.

The present disclosure also relates to a method for treating keratin materials comprising applying to the keratin materials a cosmetic composition such as defined herein.

The present disclosure still further relates to the cosmetic use of at least one diamantoid to improve the mechanical properties of at least one polymer.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific example are reported as precisely as possible. Any numerical value, however, inherently contains certain errors necessarily resulting from the standard deviation found in their respective testing measurements.

The following example illustrates the present disclosure in a non-limiting manner.

### EXAMPLE

### Example 1

### Hair Styling Composition According to the Present Disclosure

| | |
|---|---|
| Cyclopentasiloxane¹ | 10% |
| Oxyethylenated, hydrogenated castor oil (7OE)² | 10% |
| Dimethicone copolyol³ | 0.5% |
| Propylene glycol | 2.5% |
| Behentrimonium chloride⁴ | 1.2% |
| Hydroxyl substituted diamantoids⁵ | 0.01% |
| Water | qsp 100% |

¹DC 245 FLUID marketed by DOW CORNING
²ARLACEL 989 marketed by UNIQUEMA
³DC 2-5225C marketed by DOW CORNING
⁴GENAMIN KDM-F marketed by HOECHST
⁵Diamantoids such as described in the patent application WO 02/057201

What is claimed is:

1. A cosmetic composition comprising, in a cosmetically acceptable medium, at least one diamantoid comprising at least two adamantane units.

2. The cosmetic composition according to claim 1, wherein the at least two adamantane units comprise at least one heteroatom, each heteroatom replacing at least one carbon atom, and wherein the at least one heteroatom is chosen from elements of groups III, IV, V and VI of the periodic table, with the exception of carbon.

3. The cosmetic composition according to claim 1, wherein the at least one diamantoid is functionalized.

4. The cosmetic composition according to claim 2, wherein each adamantane unit comprises from 1 to 10 functional moieties, which can be the same or different.

5. The cosmetic composition according to claim 3, wherein the diamantoid is functionalized by a reaction mechanism chosen from a nucleophilic substitution, an electrophilic substitution, a free radical substitution, an addition, an elimination, a rearrangement, an oxidation, a reduction, an acido-basic reaction, an electrochemical reaction and a photochemical reaction.

6. The cosmetic composition according to claim 3, wherein the at least one diamantoid surface is functionalized by at least one moiety chosen from carboxylic acid, amine amide, ester, nitrite, hydroxy, thiol, alkyl, alkenyl, and aryl moities, and/or from oligomers, polymers and dendrimers.

7. The cosmetic composition according to claim 4, wherein the at least one functional moiety can create, with keratin fibers, at least one bond chosen from Van der Waals, hydrogen bonds, ionic bonds and covalent bonds.

8. The cosmetic composition according to claim 7, wherein the at least one functional moiety that can create at least one covalent chemical bond with keratin fibers is chosen from moieties that can react with thiols, disulfides, carboxylic acids, alcohols and amines.

9. The cosmetic composition according to claim 8, wherein the at least one functional moiety that can react with thiols, disulfides, carboxylic acids, alcohols and amines is chosen from:
epoxides,
aziridine ring moieties,
vinyl and activated vinyl moieties,
carboxylic acids and derivatives thereof,
acetals, hemiacetals,
aminals, hemiaminals,
ketones, alpha-hydroxy ketones, alpha-haloketones,
lactones, thiolactones,
isocyanate,
thiocyanate,
imines,
imides,
imidates,
oxazine and oxazoline,
oxazinium and oxazolinium,
alkyl, aryl, and aralkyl halides, the halogen being chosen from iodine, bromine or chlorine,
unsaturated ring halides, the ring being a carbon or a heterocyclic ring,
sulfonyl halides of formula RSO₂X, wherein R is an alkyl moiety and X is chosen from fluorine and chlorine, and
silicon derivatives.

10. The cosmetic composition according to claim 9, wherein the vinyl and activated vinyl moieties are chosen from acrylonitrile, acrylic and methacrylic esters, crotonic acid and ester, cinnamic acids and esters, styrene and derivatives thereof, butadiene, vinyl ethers, vinyl ketones, maleic esters, maleimides, vinyl sulfones, and vinyl cyanoacrylate.

11. The cosmetic composition according to claim 9, wherein the carboxylic acids and derivatives thereof are chosen from anhydride, acid chloride, and ester functional moieties.

12. The cosmetic composition according to claim 9, wherein the imides are chosen from succinimides and glutimides.

13. The cosmetic composition according to claim 9, wherein the succinimide is N-hydroxysuccimide ester.

14. The cosmetic composition according to claim 9, wherein the unsaturated ring halides are chosen from chlorotriazine, chloropyrimidine, chloroquinoxaline, and chlorobenzotriazole.

15. The cosmetic composition according to claim 9, wherein the silicon derivatives are chosen from alkoxy silanes and silanols.

16. The cosmetic composition according to claim 1, wherein the at least one diamantoid ranges in size from 1 nm to 1 mm.

17. The cosmetic composition according to claim 1, wherein the at least one diamantoid has a molecular weight ranging from 136 g/mol to 400 g/mol.

18. The cosmetic composition according to claim 1, wherein the at least one diamantoid is present in the cosmetic composition in an amount ranging from 0.00001% to 30% by weight, relative to the total weight of the composition.

19. The cosmetic composition according to claim 18, wherein the at least one diamantoid is present in the cosmetic composition in an amount ranging from 0.0001% to 10% by weight, relative to the total weight of the composition.

20. The cosmetic composition according to claim 19, wherein the at least one diamantoid is present in the cosmetic composition in an amount ranging from 0.001% to 5% by weight, relative to the total weight of the composition.

21. The cosmetic composition according to claim 1, further comprising at least one surfactant.

22. The cosmetic composition according to claim 21, wherein the at least one surfactant is chosen from amphiphilic molecules, amphiphilic oligomers, amphiphilic dendrimers, and amphiphilic polymers.

23. The cosmetic composition according to claim 1, wherein the cosmetically acceptable medium comprising at least one diamantoid further comprises at least one ingredient chosen from:
water,
aliphatic and/or aromatic alcohols,
volatile and/or non volatile silicones,
mineral, organic and/or vegetable oils,
oxyethylenated and/or non oxyethylenated waxes, paraffins, and alkanes,
fatty acids, fatty amines, fatty esters, and fatty alcohol benzoates or salicylates, and
acetone, methylethylketone, methyl acetate, butyl acetate, ethyl acetate, dimethoxyethane, and diethoxyethane.

24. The cosmetic composition according to claim 1, wherein the composition further comprises at least one propellant.

25. The cosmetic composition according to claim 24, wherein the at least one propellant comprises a compressed or liquefied gas chosen from air, carbon dioxide, compressed nitrogen, a soluble gas, and halogenated hydrocarbides, fluorinated or not.

26. The cosmetic composition according to claim 25, wherein said soluble gas is dimethyl ether.

27. The cosmetic composition according to claim 1, further comprising at least one cosmetic adjuvant chosen from silicones in soluble, dispersed form; thickening agents; non ionic, anionic, cationic and amphoteric surfactants; conditioning agents; softening agents; anti-foaming agents; moisturizing agents; emollient agents; plasticizers; water-soluble and oil-soluble, silicone and/or non silicone sunscreens; permanent and/or temporary dyes; inorganic and/or organic pigments, colored or not; inorganic fillers; clays; pearlescent agents; opacifying agents; colloids; fragrances; peptizing agents; preserving agents; ceramides; pseudo-ceramides; vitamins; pro-vitamins; proteins; sequestering agents; solubilizing agents; alkalinizing agents; anticorrosion agents; fats; reducing agents; antioxidants; and oxidizing agents.

28. The cosmetic composition according to claim 1, wherein the composition is a composition to be applied onto the hair and is in a form chosen from a lotion, a spray, a foam, a gel, a paste, a cream, a stick, a shampoo, an after-shampoo product, a hair repair product, a hair bleaching-composition, a perm composition, and a defrizzing composition.

29. The cosmetic composition according to claim 1, wherein the composition is a composition to be applied onto the skin and is in a form chosen from a gel, a spray, a foam, a cream, a lipstick, an eye-liner, a mascara, a blush, a nail varnish polish, a shaving foam, and a powder.

30. A method for treating keratin materials comprising applying to the keratin materials a cosmetic composition comprising, in a cosmetically acceptable medium, at least one diamantoid comprising at least two adamantane units.

31. A method for improving the mechanical properties of at least one polymer comprising adding to a composition comprising at least one polymer in a cosmetically acceptable medium, at least one diamantoid comprising at least two adamantane units.
